(19) United States
(12) Patent Application Publication
(10) Pub. No.: US 2004/0039325 A1
(43) Pub. Date: Feb. 26, 2004

(54) Treatment of blood with light

(76) Inventor: Nelson M. Karp, Virginia Beach, VA (US)

Correspondence Address: WILLIAMS MULLEN, 1 OLD OYSTER POINT ROAD, SUITE 210, NEWPORT NEWS, VA, 23602, US

(21) Appl. No.: 10/462,344

(22) Filed: Jun. 16, 2003

Related U.S. Application Data
(60) Provisional application No. 60/388,798, filed on Jun. 14, 2002.

(51) Int. Cl. A61M 37/00

(52) U.S. Cl. 604/6.08

(57) ABSTRACT

A device for the exposure of blood to light produces a controlled, safe, and rapid exposure of blood to specific emissions, thereby inducing improved immune response. The device exposes the blood through a simplified blood flow path in which the blood flow is in a spiral motion. The device includes a blood flow path, a pump, an exposure chamber, an ultraviolet light source, and a vacuum chamber.

### CROSS REFERENCE TO RELATED APPLICATION

The present application claims priority from U.S. Provisional Application Ser. No. 60/388,798, filed Jun. 14, 2002.

### BACKGROUND OF THE INVENTION

1. Field of the Invention

The present invention relates to an apparatus and method for the exposure of blood to light as a medical treatment. More specifically, the invention involves the controlled exposure of a stream of blood to ultraviolet (UV) light under conditions that effect a salubrious change in the blood.

2. Description of the Related Art

Light is well known as an effective medical treatment. In the nineteenth century, it was shown that light could inhibit bacteria growth and kill some microorganisms. In 1903, Niels R. Finsen won the Nobel Prize for Medicine by treating certain skin conditions (e.g., tuberculosis) with light. Before the advent of antibiotics, physicians began to use UV light to treat a variety of infections, many of which were ill defined at the time.

In the 1920's, 1930's, and 1940's, researchers began to develop devices for the exposure of blood to UV radiation, and reported positive results. For example, U.S. Pat. Nos. 1,683,877, 2,308,516, and 2,309,124 to L. A. Edblom and E. K. Knott are considered by some to be the key, early developments in the field, which was known as the Knott HEMOIRRADIATOR®. Although the mechanism of this device and the treatment were little understood at the time, the conclusion was that UV blood irradiation therapy enhanced the body's immune response. These references disclosed an extra-corporeal system in which whole blood was drawn, mixed with an anti-coagulant, pumped through a chamber where it was exposed to UV light between 1800 and 4000 angstroms—with a concentration or peak at 2540 angstroms—and then returned to the body. Although the first invention disclosed blood flow through two needles, Knott found a single needle arrangement to be speedier for his closed loop system. The exposure chamber included a transparent window through which the light source would shine onto the flowing blood. The chamber was designed to agitate the blood as it flowed by this flat window, so that more cells and bacteria would be exposed to the UV light. The '516 patent refined the device and advised users that exposure of more than 5 seconds could be detrimental.

Historically, the introduction of antibiotics and vaccines reduced the interest in the use of light for medical treatment. Nevertheless, development continued throughout the twentieth century. Most of the later developments seemed to be characterized by inventions that involved: (a) the separation and exposure of a portion or component of the blood (e.g., U.S. Pat. No. 4,613,322 to Edelson), (b) the addition of a compound or photo-active agent to the blood (e.g., U.S. Pat. No. 4,737,140 to Lee), or (c) both (e.g., U.S. Pat. Nos. 4,321,919, 4,398,906, 4,464,166, 4,612,007, 4,613,322, 4,683,889, 4,684,521 all to Edelson). A few references addressed improvements in the design of the exposure chamber and the blood transport system. As in the Knott design, all these developments retained the need for anti-coagulant treatment, and focused on large or macro scale improvements in exposure.

In U.S. Pat. No. 5,150,705, Stinson disclosed a cylindrical exposure chamber comprising a central UV light source, effective for treating transplant cells, located within a UV transparent cylinder, and UV transparent tubing for carrying a cellular suspension. The tubing is wrapped helically about the cylinder to promote consistent exposure of the tubing to the UV source. Such a macro scale arrangement was intended to maximize the tube's efficiency in capturing UV emissions from a cylindrical source. However, depending on the overall fluid characteristics, the micro scale blood flow relative to the tube could be stratified by density, leading to uneven exposure of the blood.

Another example of an evolutionary configuration of an exposure chamber is shown in U.S. Pat. No. 6,312,593, to Petrie. The Petrie device discloses a chamber that features a series of baffle plates and transverse protuberances to produce a Bernoulli distortion in the blood flow, creating desirable agitation. In fact, the Knott design contemplated agitation of the blood; this invention used the energy of the fluid flow reacting to a transverse disturbance to cause a pressure gradient, thereby forcing blood at a lower depth to move upward. As the blood moved up, it had a greater chance of being exposed to the flat transparent window that Knott introduced. The transverse aspect of the disturbance required sufficient longitudinal flow to generate enough disturbance for the desired exposure.

Accordingly it is an object of the present invention to provide a method for treating blood which provides improved exposure of blood to UV light.

Another object of the present invention is to provide a method for treating blood which increases the likelihood that each blood cell is exposed to the proper amount of UV light.

Another object of the present invention is to provide a method for treating blood which reduces the amount time blood must be exposed to UV light.

Another object of the present invention is to provide a method for treating blood which reduces the likelihood of cellular separation and hemolysis.

Yet another object of the present invention is to provide a method for treating blood which treats blood rapidly.

Yet another object of the present invention is to provide a method for treating blood which does not result in coagulation of the blood.

Finally, it is an object of the present invention to accomplish the foregoing objectives in a simple and cost effective manner.

### SUMMARY OF THE INVENTION

The present invention addresses the foregoing problems, as well as other problems, by providing a chamber for exposing a stream of blood flowing through the chamber to ultraviolet light to destroy microorganisms and to stimulate the immune system. The chamber includes an ultraviolet-light-transparent blood flow path having an inlet port and an outlet port. The blood is induced to flow in spiral flow within the chamber and exposed to an ultraviolet light source. The blood flow path can be formed from any ultraviolet-light-transparent material such as polystyrene, polypropylene or quartz. If desired, the blood flow path can be formed from a plurality of materials, such that different portions absorb a different wavelength of UV light or, alternatively, one or more filters can be used to regulate the exposure of the blood flow path to ultraviolet rays. The spiral movement in the blood is caused by, for example, twisting the blood flow path, by forming threaded walls on the internal surface of the blood flow path or by an agitation means. Preferably, a vacuum is formed between the ultraviolet light source and the blood flow path. Any gaps between the ultraviolet light source and the blood flow path can be filled with an optically transparent material such as quartz. The ultraviolet light source can be one or more light bulbs, preferably emitting light within at least the ultraviolet A range. If appropriate, the ultraviolet light can be pulsed such that the blood is exposed to the ultraviolet light on a discontinuous basis.

### DESCRIPTION OF THE DRAWINGS

FIG. 1 is a view of the flow path used in the preferred embodiment of the present invention;

FIG. 2 is a diagram showing the method of using the preferred embodiment of the present invention; and

FIG. 3 is a diagram showing a particularly preferred embodiment of the present invention.

### DETAILED DESCRIPTION OF THE PREFERRED EMBODIMENT

The following detailed description is of the best presently contemplated modes of carrying out the invention. This description is not to be taken in a limiting sense, but is made merely for the purpose of illustrating general principles of embodiments of the invention.

A device for the exposure of blood to light can be built that produces a controlled, safe, and rapid exposure of blood to specific emissions, thereby inducing improved immune response. The present invention achieves this through a simplified blood flow path. On a macro scale, the invention includes a blood flow path, a pump, an exposure chamber, a light source, and a vacuum chamber. The preferred embodiment contemplates an UV light source. Preferably, the invention is of a flow-through design.

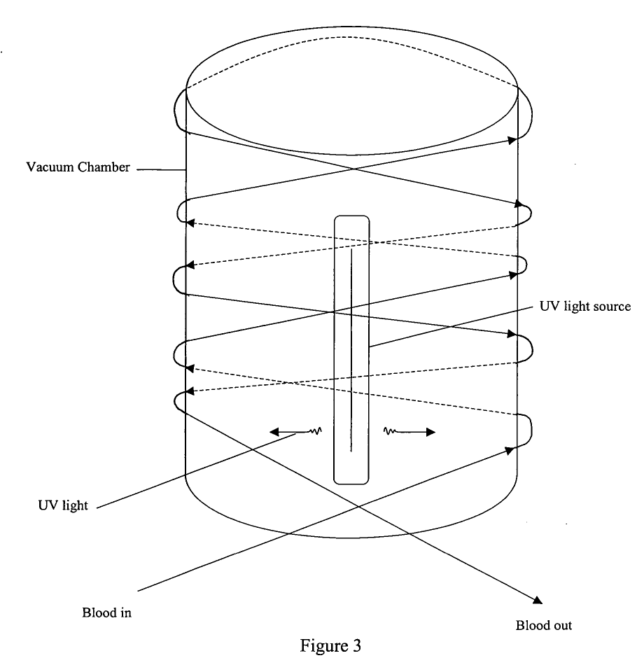

The exposure chamber design enhances control over the exposure of the blood to ultraviolet light. The exposure chamber includes a blood flow path, tube, or channel crafted from a UV transparent material, as is available commercially in transparent polystyrene or polypropylene. These materials permit blood flow in a variety of configurations or arrangements during the period of exposure to UV light, so long as each red blood cell is exposed to the emissions for the desired time. In the event that an otherwise desirable transparent material is found to absorb an important wavelength of UV light, then the flow path could be constructed of segments of varying light absorption characteristics (i.e., different transparent materials). Alternatively, filters may be employed to expose the blood to selected wavelengths. A particularly preferred design for the exposure chamber is a double helix type geometry as shown in FIG. 3. In this embodiment, the UV light source is positioned in the center of the chamber. This design results in particularly consistent and uniform exposure of the blood to the UV light and minimization of hemolysis.

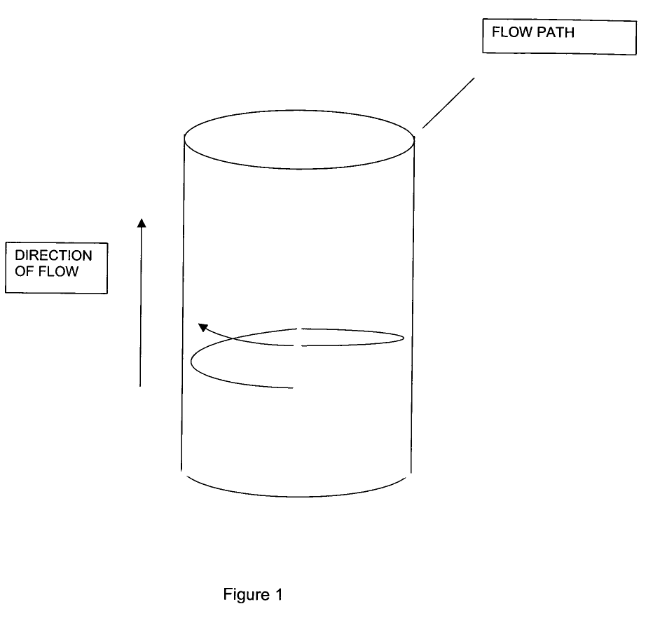

Consistent exposure of all or most of the subject cells is safer and more efficient than variable, partial exposure. A spiral or helical path of tubing on a macro scale has been tried in order to enable the tube to embrace a cylindrical UV source and to capture efficiently its emissions. That design concentrated on efficient use or capture of the UV emissions. However, a spiral flow path on a micro scale can produce the internal flow characteristics and external chamber geometry useful to enhance micro scale control. In the present invention, the blood flow is rendered helical or spiral within the channel, as shown in FIG. 1, and then the channel can be configured about an ultraviolet light source for the desired exposure.

The desired internal helix or spiral may be achieved in a variety of ways. Aside from macro scale orientation or twisting of a flow channel or tube to induce a micro scale spiral or helix flow, the channel may feature internally threaded walls or include an internal Kenics static mixer. The internal production of a spiral flow is adaptable to 360-degree exposure, either directly or through reflection. The frequency of the spiral rotation may be segmented or metered for the desired exposure, at a given a chamber size, source, and flow rate. Although different applications may drive a final configuration, such a spiral flow enables greater control of the exposure to of the sample volume. The blood cells are spiraled or mixed internally during their longitudinal travel along the flow path, and are therefore more likely to be exposed to the UV emissions during their transit of a given length of flow path. A predictable level of exposure per longitudinal unit means that the overall time of exposure may be reduced. Those skilled in the art of blood flow dynamics will readily see the alternative configurations available.

Preferably, for the macro scale design, any space (preferably less than 3 cm) between the blood and the ultraviolet light source is filled with an optically transparent material, such as quartz. Further, preferably gaps or voids between the blood flow path and the ultraviolet light source should be maintained at a vacuum to avoid ionization of the air and variation in the characteristics of the radiation.

The light source should provide the desired spectral emissions, with exposure appropriate to the flow and configuration of the chamber. The UV light source can be a single UV light bulb or a plurality of UV light bulbs. Preferably, a light source emitting UVA light is used. If a light bulb, or combination of light bulbs, which emits UVB or UVC light in addition to UVA light is used, one or more filters can be used so that the blood is exposed to the desired UVA light. Ideally, the blood is exposed to UV light having peak wavelengths of 365 nm and 254 nm. Designs based on the Knott device would provide a range of UV wavelengths from 2000 to 4000 angstroms, with an intensity ranging from 40 to 1,538 W/cm². Other designs, such as that in '566 to Schleicher, contemplate a wavelength output of 2,000 to 12,000 angstroms. The UV light source may be pulsed or shuttered at a desired frequency; in this case, a pulse of approximately two to three hertz would be standard. As noted above, micro scale induction of spiral flow enables a wide variety of macro scale arrangements, so the blood flow path can be structured to accommodate different types of light sources. To ensure that the blood is being exposed to the appropriate UV light, a spectrophotometer can be used to monitor and regulate, when appropriate, the UV emissions.

The design of the exposure chamber, the pulse frequency and intensity, and the volumetric flow rate are preferably considered together for optimal control. Many whole blood designs seek an individual red cell exposure period of about ¼ to 1/2 seconds, while also avoiding cellular separation and hemolysis. The volume of the exposure chamber will depend on the pulse frequency, desired exposure time, and volumetric flow rate. A sample of approximately 250 milliliters of blood (or 1.5 milliliters per kilogram of body weight) may be treated at a time. In a design with a volumetric blood flow rate of approximately 1 milliliter per second, the device could process the entire sample through the exposure chamber in a period of four minutes and ten seconds.

In a particularly preferred embodiment, the exposure chamber and blood flow path can be made as disposable items. This embodiment has the added benefits of eliminating the need to sterilize these items and the concern of transmission of infectious diseases.

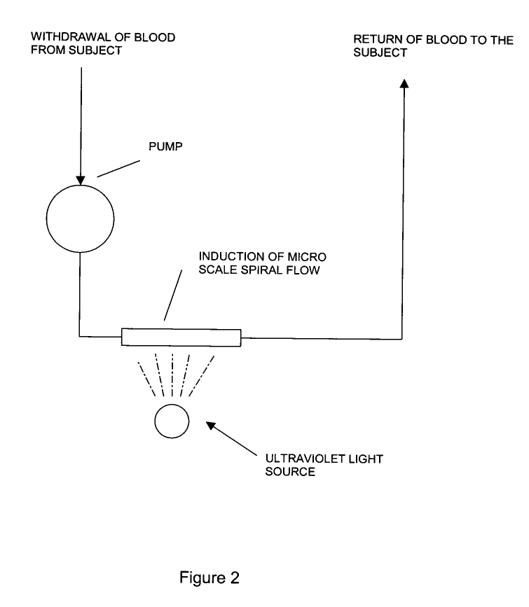

An additional feature of the micro scale approach is reduced time of treatment. In the present invention, the spiral flow within the exposure chamber promotes controlled, thorough, and rapid exposure. Prior designs that operated more slowly face the problem of coagulation of the blood. Typical approaches to compensate for this problem are the addition of heparin, or the inclusion of other anti-coagulant measures. The rapidity and control of the present invention offers an alternative. Preferably, the blood should be drawn from one point and returned to the patient at a different point, as shown in FIG. 2. For example, the blood could be withdrawn from the antecubital fossa or other convenient venous access of the upper extremity of a left arm and, after treatment, returned to a symmetric location on the right arm. This quick, flow through design, combined with rapid exposure to the light source; reduces the chance of coagulation of the blood, so that an anti-coagulant step is not required. In fact, in its whole blood treatment embodiment, the present invention contemplates no need for any other additives, such as photopheretic compounds or other active agents.

In an alternate embodiment of the present invention, the blood can be separated into four portions once it is removed from the patient: erythrocytes, leukocytes, platelets and blood plasma. In this embodiment, each portion would be separately exposed to ultraviolet light for treatment and then reintroduced to the patient separately or collectively.

This contemplated arrangement for the exposure of blood to light may be achieved in a variety of configurations. While there has been described what are believed to be the preferred embodiment of the present invention, those skilled in the art will recognize that other and further changes and modifications may be made thereto without departing from the spirit of the invention, and it is intended to claim all such changes and modifications as fall within the true scope of the invention.

What is claimed is:

1. A chamber for exposing a stream of blood flowing through the chamber to ultraviolet light to destroy microorganisms and to stimulate the immune system; said chamber comprising:
an ultraviolet-light-transparent blood flow path having an inlet port and an outlet port;
a means located within said blood flow path for inducing a spiral movement in the blood as said blood flows from the inlet port to the outlet port along the blood flow path; and
an ultraviolet light source adapted to expose the blood flow path with ultraviolet rays.

2. The chamber described in claim 1 wherein the blood flow path is formed from a material selected from the group consisting of polystyrene, polypropylene and quartz.

3. The chamber described in claim 1 wherein the blood flow path is formed from a plurality of materials, each material absorbing a different wavelength of UV light.

4. The chamber described in claim 1 further comprising at least one filter for regulating the exposure of the blood flow path to ultraviolet rays.

5. The chamber described in claim 1 wherein the spiral movement in the blood is caused by twisting the blood flow path.

6. The chamber described in claim 1 wherein the spiral movement in the blood is caused by forming threaded walls on the internal surface of the blood flow path.

7. The chamber described in claim 1 wherein the spiral movement in the blood is caused by an agitation means.

8. The chamber described in claim 1 wherein a vacuum is formed between the ultraviolet light source and the blood flow path.

9. The chamber described in claim 1 wherein there is a gap between the ultraviolet light source and the blood flow path and the gap is filled with an optically transparent material.

10. The chamber described in claim 10 wherein the optically transparent material is quartz.

11. The chamber described in claim 1 wherein the ultraviolet light source consists of an ultraviolet light bulb emitting light in the ultraviolet A range.

12. The chamber described in claim 1 wherein the ultraviolet light source consists of a plurality of ultraviolet light bulbs.

13. The chamber described in claim 1 wherein the ultraviolet light source is pulsed such that the blood is exposed to the ultraviolet light on a discontinuous basis.

14. A method for treating blood with ultraviolet light, comprising:
removing blood from a patient at a first location;
pumping the blood through an exposure chamber wherein the blood flows in a spiral motion within a blood flow path;
exposing the blood flow path to an ultraviolet light source; and
returning the exposed blood to the patient at a second location.

15. The method for treating blood described in claim 15 wherein the blood flow path is formed from an ultraviolet light transparent material.

16. The method for treating blood described in claim 15 wherein the spiral motion of the blood flow is induced by twisting the blood flow path.

17. The method for treating blood described in claim 15 wherein the spiral motion of the blood flow is induced by forming threaded walls on the internal surface of the blood flow path.

18. The method for treating blood described in claim 15 wherein the spiral motion of the blood flow is induced by an agitation means.

19. The method for treating blood described in claim 15 wherein a vacuum is formed between the ultraviolet light source and the blood flow path.

20. The method for treating blood described in claim 15 wherein there is a gap between the ultraviolet light source and the blood flow path and the gap is filled with an optically transparent material.
